(19) United States
(12) Patent Application Publication
(10) Pub. No.: US 2008/0103508 A1
(43) Pub. Date: May 1, 2008

(54) APPARATUS AND METHOD FOR REMOVAL OF FOREIGN MATTER FROM A PATIENT

(76) Inventor: Ali Serdar Karakurum, Jericho, NY (US)

Correspondence Address: ANTHONY R. BARKUME, 20 GATEWAY LANE, MANORVILLE, NY, 11949, US

(21) Appl. No.: 11/555,316

(22) Filed: Nov. 1, 2006

(51) Int. Cl. A61B 17/22

(52) U.S. Cl. 606/127

(57) ABSTRACT

Apparatus for removal of a bolus of foreign matter such as impacted food from an esophagus; including an overtube with an overtube insertion end and an overtube outside end for insertion into an esophagus. A substantially cylindrical basket has a first basket end adjoined to the overtube insertion end. A wire sleeve is inserted within the overtube, having a wire sleeve insertion end disposed near the overtube insertion end and a wire sleeve outside end disposed outside the overtube outside end. A wire is slidingly inserted within the wire sleeve and terminates in a snare loop disposed outside the wire sleeve and integrated with the second basket end. A wire position control is integrated with the wire sleeve outside end and adapted to extend and retract the wire with respect to the wire sleeve as desired.

### TECHNICAL FIELD

This invention relates to the removal of foreign matter, and in particular to the removal of foreign matter such as a food bolus from the esophagus of a human being.

### BACKGROUND ART

Esophageal food impaction is a common problem in the field of gastroenterology. Current treatment procedures involve an urgent endoscopy in an attempt to either push the impacted food (referred to as a bolus) into the patient's stomach via an endoscope, or to remove the impacted food bolus with a snare or forceps using an overtube to prevent aspiration.

There are several problems with these prior art methods. Often there is a stricture (i.e. narrowing) distal to the impacted food bolus that presents a risk of further impaction or perforation of the esophagus since the degree of stricture is unknown. Removal of a food bolus using a snare or biopsy forceps (i.e. a device known as a Rotatable Retrieval Basket from US ENDOSCOPY) often results in the breaking up of the food bolus, making it very difficult to remove the bolus in small pieces. This will likely prolong the extraction procedure and add excessive risks to the procedure.

A device known as a ROTH NET, available from US ENDOSCOPY, uses a wire snare and a netting material designed to scoop foreign materials such as coins, watch batteries and the like, from a stomach. This type of device requires scooping of a loose object and is not suitable for encasing and capturing a food bolus that has been impacted in the esophagus.

Thus, it is an object of the invention to provide an apparatus and method if use of the apparatus for successful removal of a food bolus from the esophagus with the risks described above.

### DISCLOSURE OF THE INVENTION

The present invention is an apparatus for removal of a bolus of foreign matter from an esophagus. The apparatus has an overtube suitable for insertion into an esophagus, the overtube including an overtube insertion end and an overtube outside end. A substantially cylindrical basket has a first basket end adjoined to the overtube insertion end and a second basket end opposite the first basket end. A wire sleeve is inserted within the overtube, the wire sleeve having a wire sleeve insertion end and a wire sleeve outside end, the wire sleeve insertion end being disposed near the overtube insertion end and the wire sleeve outside end being disposed outside the overtube outside end. A wire is slidingly inserted within the wire sleeve, the wire terminating in a snare loop disposed outside the wire sleeve and integrated with the second basket end. A wire position control is integrated with the wire sleeve outside end and adapted to extend and retract the wire with respect to the wire sleeve as desired. As such, when the wire is extended to a first position by the wire position control, the snare loop allows the second basket end to be in an open position, and when the wire is retracted to a second position by the wire position control, the snare loop is pulled towards the wire sleeve insertion end and urges the second basket end towards a closed position, thus closing the basket.

An endoscope assembly including a camera and a light is slidingly inserted within the overtube and is adapted to enable images to be obtained from a region outside of the overtube insertion end. Use of the endoscope enables the doctor or other professional operating the removal apparatus to guide the basket over the bolus as follows. In order to remove the foreign body of matter (the bolus), the operator will implement the wire position control in order to extend the wire to the first position and cause the snare loop to allow the second basket end to be in an open position. Then the overtube is inserted into the esophagus with the second basket end in the open position. The camera and light at the end of the endoscope will enable images to be viewed by the operator and help him or her to guide the second basket end over the bolus until the basket substantially surrounds the bolus. The operator will then implement the wire position control in order to retract the wire to the second position and cause the snare loop to be pulled towards the wire sleeve insertion end and urge the second basket end towards a closed position, thus encasing the bolus within the basket. After the bolus has been encased within the basket, the operator will withdraw the overtube and attached basket from the esophagus in order to remove the bolus therefrom.

### BRIEF DESCRIPTION OF THE DRAWING

FIG. 1 is an illustration of the preferred embodiment apparatus of the present invention with an endoscope extended therefrom, just prior to ensaring a food bolus in an esophagus.

FIG. 2 is an illustration of the apparatus of FIG. 1 with the basket in a closed position and the target food bolus captured within.

FIG. 3 is a perspective drawing of the basket of the present invention.

### BEST MODE FOR CARRYING OUT THE INVENTION

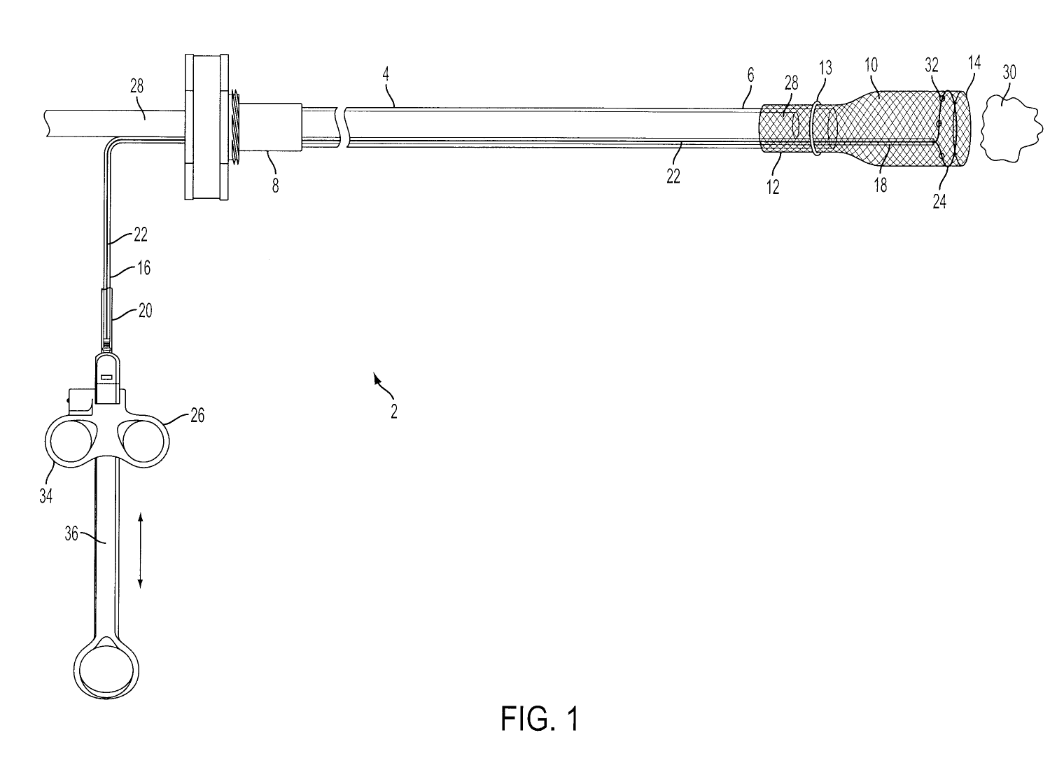

The preferred embodiments of the present invention will now be described with respect to the Figures. FIG. 1 illustrates a preferred embodiment of the present invention. The apparatus 2 includes an overtube 4, which is adapted to be slidingly inserted into a patient's esophagus in order to remove a food bolus 30 that has become lodged therein. The overtube is a thin, flexible lighted tube as known in the art and is typically used for insertion into the esophagus for performing an endoscopy. For example, an overtube such as one known as the GUARDUS OVERTUBE from US ENDOSCOPY may be implemented as described herein. An endoscope 28 is mounted through the overtube 4 as known in the art, and has a camera and light mounted for obtaining images of the internal area under surveillance and assisting the operator in guiding the overtube (not shown for simplicity).

The overtube 4 has an overtube insertion end 6, which is the end that will be inserted into the esophagus of the patient. The opposite end of the overtube is the overtube outside end 8, into which the endoscope may be inserted. As shown in FIG. 1, a basket 10, which is substantially cylindrical in shape, is adjoined by its first basket end 12 to the overtube insertion end 6. The diameter of the basket may be approximately the same as the diameter of the overtube in order to provide for easier insertion into the patient. An elastic band 13 urges the basket against the endoscope and prevents leakage therethrough. The basket 10 also has a second basket end 14 opposite the first basket end 12, the operation of which will be fully described below.

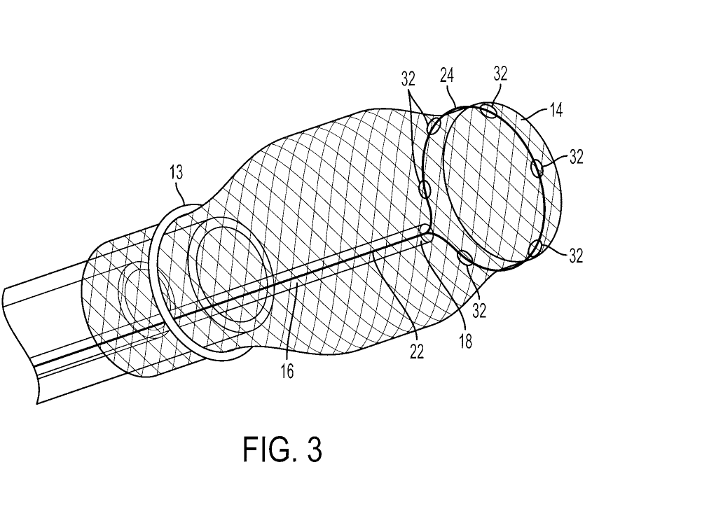

A wire sleeve 16, which is a thin flexible plastic tube, is inserted within the overtube 4 and extends through the entire body of the overtube 4. A wire sleeve insertion end 18 is located near the overtube insertion end (see FIG. 3 as well). The wire sleeve 16 also has a wire sleeve outside end 20, which is integrated with a wire position control device 26 as further described below.

Inserted through the wire sleeve 16 is a wire 22. The wire extends through the length of the wire sleeve 16, and terminates in a snare loop 24 at the end that extends outside of the wire sleeve insertion end 18 as shown in FIG. 3. The snare loop 24 is integrated with the second basket end 14, typically by looping around it near the end thereof. In particular, in the preferred embodiment, the snare loop is cinched to the second basket end in a belt-like manner. In this case, a series of small loops 32 are used to loosely secure the snare loop around the perimeter of the second basket end 14, allowing the snare loop 24 to draw closed and thusly close the second basket end 14 as further described below. A snare similar to the ROTATOR Rotatable Polypectomy Snare, available from US ENDOSCOPY, may be suitable for use with the present invention.

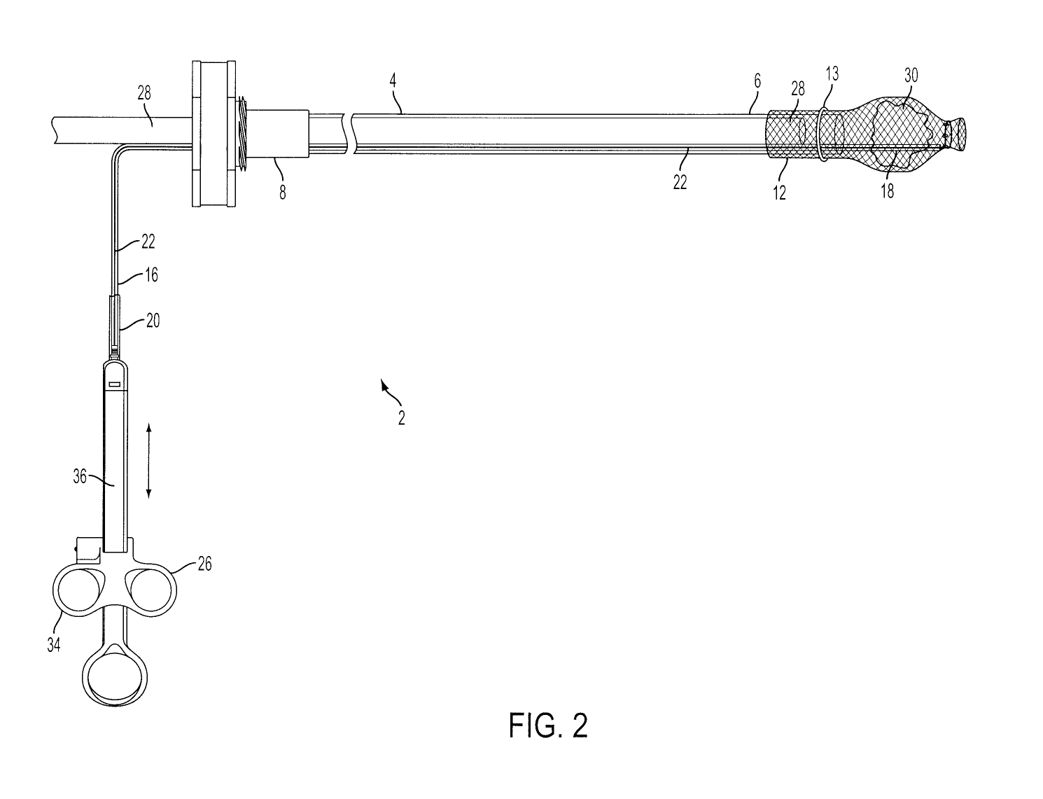

The wire position control 26 is located at the wire sleeve outside end. The wire position control interoperates with the wire 22 and the wire sleeve outside end 20 in order to allow the operator to slide the wire back and forth within the wire sleeve 16; either extending the wire to a first position (as shown in FIG. 1) or retracting the wire to a second position (as shown in FIG. 2), as desired. When the wire is extended to a first position by the wire position control, the snare loop 24 is in a relaxed state and it allows the second basket end 14 to maintain its naturally open position, as shown in FIG. 1. When, however, the wire is retracted to the second position, the snare loop 24 is caused to be drawn into the wire sleeve insertion end 18 as the wire is retracted, and the second basket end 14 is, as a result, caused to close as shown in FIG. 2.

The wire position control 26 used in the preferred embodiment has a handle 34 and a push rod 36 suitable for gripping and squeezing in order to retract and extend the wire as described. For example, the wire position control 26, wire sleeve 16, wire 22, and snare loop 24 operate in a manner similar to the ROTATOR snare available from US ENDOSCOPY. Other types of wire control devices may be used in order to cause the wire to extend and retract in accordance with the principles of the present invention.

The operation of the above-described apparatus is as follows. An endoscope assembly 28 including a camera and a light (not shown) is slidingly inserted within the overtube 4 and is adapted to enable images to be obtained from a region outside of the overtube insertion end 8 (i.e. within the vicinity of the bolus 30). Use of the endoscope enables the doctor or other professional operating the removal apparatus to guide the basket over the bolus as follows. In order to remove the bolus 30, the operator will implement the wire position control 26 in order to extend the wire 22 to the first position and cause the snare loop 24 to allow the second basket end 14 to be in an open position. Then, the overtube 4 is inserted into the esophagus with the second basket end 14 in the open position. The camera and light at the end of the endoscope 28 will enable images to be viewed by the operator and help him or her to guide the second basket end 14 over the bolus until the basket 10 substantially surrounds the bolus 30. The operator will then implement the wire position control 26 in order to retract the wire 22 to the second position and cause the snare loop 24 to be pulled towards the wire sleeve insertion end 18, tighten around the loops 32, and urge the second basket end 14 towards a closed position, thus encasing the bolus 30 within the basket 10. After the bolus 30 has been encased within the basket, the operator will withdraw the overtube 4 and attached basket 10 from the esophagus in order to remove the bolus therefrom.

The basket should be fabricated from a material that will be resilient enough to keep its normal (open) shape when the snare loop is relaxed by the operator, as well as to be able to be forced around the bolus that is lodged in the esophagus. The material must also be pliable enough to be allowed to be closed by the operation of the snare after the basket has surrounded the bolus. In a preferred embodiment, the basket may be fabricated from a silicone material with a polyester mesh, such as the material used for a POLYFLEX stent available, for example, from BOSTON SCIENTIFIC. In addition, a metal stent material may be used, such as the wall stent made by BOSTON SCIENTIFIC and COOK. Other materials may be used as long as they exhibit the required resilient and pliable qualities as described herein. The basket may be covered by a membrane material if desired.

It will be apparent to those skilled in the art that modifications to the specific embodiment described herein may be made while still being within the spirit and scope of the present invention

What is claimed is:

1. An apparatus for removal of a bolus of foreign matter from an esophagus comprising:
a. an overtube suitable for insertion into an esophagus, the overtube comprising an overtube insertion end and an overtube outside end;
b. a substantially cylindrical basket comprising a first basket end and a second basket end, the first basket end adjoined to the overtube insertion end;
c. a wire sleeve inserted within the overtube, the wire sleeve comprising a wire sleeve insertion end and a wire sleeve outside end, the wire sleeve insertion end being disposed near the overtube insertion end and the wire sleeve outside end being disposed outside the overtube outside end;
d. a wire slidingly inserted within the wire sleeve, the wire terminating in a snare loop disposed outside the wire sleeve and integrated with the second basket end; and
e. a wire position control integrated with the wire sleeve outside end and adapted to extend and retract the wire with respect to the wire sleeve as desired;
whereby, when the wire is extended to a first position by the wire position control, the snare loop allows the second basket end to be in an open position, and when the wire is retracted to a second position by the wire position control, the snare loop is pulled towards the wire sleeve insertion end and urges the second basket end towards a closed position.

2. The apparatus of claim 1 further comprising
f. an endoscope assembly slidingly inserted within the overtube; the endoscope assembly adapted to enable images to be obtained from a region outside of the overtube insertion end.

3. The apparatus of claim 1 wherein the wire position control is integrated with the wire sleeve outside end and adapted to extend and retract the wire with respect to the wire sleeve with a plurality of loops attached to the second basket end into which the snare loop is loosely inserted.

4. The apparatus of claim 1 wherein the basket comprises a silicone material.

6. The apparatus of claim 1 wherein the basket comprises a metal material.

7. The apparatus of claim 1 wherein the basket is covered by a membrane.

8. A method of extracting a bolus of foreign matter from an esophagus utilizing the apparatus of claim 2 comprising the steps of:
i) implementing the wire position control in order to extend the wire to the first position and cause the snare loop to allow the second basket end to be in an open position;
ii) inserting the overtube into the esophagus with the second basket end in the open position;
ii) guiding the second basket end over the bolus until the basket substantially surrounds the bolus;
iii) implementing the wire position control in order to retract the wire to the second position and cause the snare loop to be pulled towards the wire sleeve insertion end and urge the second basket end towards a closed position, thus encasing the bolus within the basket; and
iv) withdrawing the overtube from the esophagus in order to remove the bolus therefrom.
